(19) United States
(12) Patent Application Publication
(10) Pub. No.: US 2026/0245538 A1
(43) Pub. Date: Aug. 20, 2026

(54) AUDIO SPINAL SIGNAL

(71) Applicant: Medtronic, Inc., Minneapolis, MN (US)

(72) Inventors: Leonid M. Litvak, Bet Shemesh (IL); David A. Dinsmoor, North Oaks, MN (US); Jerel K. Mueller, St. Paul, MN (US); Joshua J. Nedrud, Roseville, MN (US); Jeffery M. Kramer, St. Louis Park, MN (US); Ashwini Dayal Sharan, Pennington, NJ (US)

(21) Appl. No.: 19/161,631

(22) PCT Filed: Mar. 11, 2024
(86) PCT No.: PCT/IB2024/052333

§ 371 Date: Sep. 2, 2025

Related U.S. Application Data
(60) Provisional application No. 63/491,491, filed on Mar. 21, 2023.

(51) Int. Cl. G10K 15/02; A61N 1/372

(52) U.S. Cl. CPC G10K 15/02; A61N 1/37247

(57) ABSTRACT

Devices, systems and techniques for receiving physiological signals from a patient, encoding the signals into an audio representation of the signal and outputting and audio signal for a user. The encoding of this disclosure may combine, separate, exaggerate and otherwise process the received signals to generate an audio signal that a user, such as a clinician or other caregiver, or the patient, may evaluate and decide whether to take some action based on the audio signal. In some example, the physiological signals, or portions of the physiological signals may also be displayed on a display device. In some examples, the encoding may improve audio contrast of components of the received physiological signal by removing background and accentuating a signal of interest.

This Application claims priority from U.S. Provisional Patent Application 63/491,491, filed 21 Mar. 2023, the entire content of which is incorporated herein by reference.

### TECHNICAL FIELD

The disclosure relates to user interfaces for sensed bioelectrical signals from a patient.

### BACKGROUND

Medical devices may be external or implanted and may be used to measure, analyze, and present to a clinician, or a patient, bioelectrical signals from the patient. The medical devices may also deliver electrical stimulation to patients via various tissue sites to treat a variety of symptoms or conditions such as chronic pain, tremor, Parkinson's disease, epilepsy, urinary or fecal incontinence, sexual dysfunction, obesity, or gastroparesis. A medical device may deliver electrical stimulation therapy via one or more leads that include electrodes located proximate to target locations associated with the brain, the spinal cord, pelvic nerves, peripheral nerves, or the gastrointestinal tract of a patient. Stimulation proximate the spinal cord, proximate the sacral nerve, within the brain, and proximate peripheral nerves are often referred to as spinal cord stimulation (SCS), sacral neuromodulation (SNM), deep brain stimulation (DBS), and peripheral nerve stimulation (PNS) including tibial nerve stimulation (TNS), respectively. Electrical stimulation may be delivered by the medical device as a train of pulses, and the values of the parameters defining the pulses may be altered.

### SUMMARY

In general, the disclosure describes devices, systems and techniques for receiving physiological signals from a patient, encoding the signals into an audio representation of the signal and outputting and audio signal for a user. The encoding of this disclosure may combine, separate, exaggerate and otherwise process the received signals to generate an audio signal that a user, such as a clinician or other caregiver, or the patient, may evaluate and decide whether to take some action based on the audio signal. In some examples, the physiological signals, or elements of the physiological signals may also be displayed on a display device, such as a screen of a programmer or other user device. In some examples, the encoding may improve audio contrast of elements of the received physiological signal by removing background and accentuating a signal of interest.

In one example, this disclosure describes a system comprising a memory; and processing circuitry operatively coupled to the memory, the processing circuitry configured to: receive, from sensing circuitry, information representative of a physiological signal from a patient, wherein the information represents a plurality of physiological elements contained in the physiological signal; receive a selection of at least one physiological element of the plurality of physiological elements; encode the selected at least one physiological element of the physiological signal into an audio representation; and control audio output circuitry to output the audio representation, wherein the audio representation is configured to be perceivable by a human ear.

In another example, this disclosure describes a method comprising receiving, by processing circuitry of a medical device and from sensing circuitry, information representative of a physiological signal from a patient, wherein the information represents a plurality of physiological elements contained in the physiological signal; receiving a selection of at least one physiological element of the plurality of physiological elements encoding, by the processing circuitry, the selected at least one physiological element of the physiological signal into an audio representation; and controlling audio output circuitry to output the audio representation, wherein the audio representation is configured to be perceivable by a human ear.

In another example, this disclosure describes a non-transitory computer-readable storage medium comprising receive, from sensing circuitry of a medical device, an indication of a physiological signal from a patient, wherein the physiological signal comprises an evoked compound action potential (ECAP); receive a selection of at least one physiological element of the plurality of physiological elements; encode the selected at least one physiological element of the physiological signal into an audio representation; and control audio output circuitry to output the audio representation, wherein the audio representation is configured to be perceivable by a human ear.

The details of one or more examples of the disclosure are set forth in the accompanying drawings and the description below. Other features, objects, and advantages of the disclosure will be apparent from the description and drawings, and from the claims.

### BRIEF DESCRIPTION OF DRAWINGS

FIG. 1 is a conceptual diagram illustrating an example system that includes an implantable medical device (IMD) configured to measure bioelectrical signals for spinal cord stimulation (SCS) therapy and an external computing device, in accordance with one or more techniques of this disclosure.

FIG. 2 is a block diagram illustrating an example combination of components of an IMD, in accordance with one or more techniques of this disclosure.

FIG. 3 is a block diagram illustrating an example combination of components of an example external computing device, in accordance with one or more techniques of this disclosure.

FIG. 4 is a block diagram illustrating an example of system components involved in outputting the audio signals described in this disclosure.

FIGS. 5A and 5B are conceptual diagrams illustrating aggressors that may impact a measured signal.

FIG. 6 is a timing diagram illustrating an example stimulation delivery and measured response according to one or more techniques of this disclosure.

FIG. 7 is a time graph illustrating how changes in the stimulation current may affect the amplitude of an ECAP.

FIG. 8 is a time graph illustrating an example of adjusting the stimulation output and the effect the ECAP.

FIG. 9 is a time graph illustrating an example multi-channel representation of physiological signals.

FIG. 10 is a flowchart illustrating an example operation of the audio generation system of this disclosure.

### DETAILED DESCRIPTION

The disclosure describes devices, systems and techniques for receiving physiological signals from a patient, encoding the signals into an audio representation of the signal, and outputting and audio signal for a user. Medical devices and systems may sense, record and present physiological information from a patient to a caregiver, such as a clinician, and in some examples to the patient. Physiological information, such as blood pressure, heart activity, and oxygen saturation, may be presented as a printout, as a report, a bar chart or similar chart, displayed on a display device including a screen, a dial or some other visual representation of the information.

The encoding of this disclosure may combine, separate, exaggerate and otherwise process the received signals to generate an audio signal that a user, such as a clinician or other caregiver, or the patient, may evaluate and decide whether to take some action based on the audio signal. In some examples, the physiological signals, or elements of the physiological signals may also be displayed on a screen to be output together with the audio signal. In some examples, the encoding may improve, at least to the perception of the user, audio contrast of elements of the received physiological signal by removing background and accentuating a signal of interest.

Some examples of elements of the physiological signals may include an evoked compound action potential (ECAP), electromyogram (EMG), cardiac activity, patient respiration, impedance, and similar bioelectrical signals measured by electrodes in contact with target tissue of the patient. Other physiological elements may include patient movement and posture, e.g., measured by an accelerometer, patient temperature, blood pressure, cranial pressure or other similar signals. In some examples, users can listen to audio representing measured physiological elements without looking at the screen, and make clinical decisions regarding the presence of an ECAP, and how much the ECAP may change with aggressors, effectiveness of aggressor management such as a closed loop response of a medical device, latency of an ECAP, or any other clinically relevant observations. The user can then use this information for a variety of purposes, such as determining lead location during placement surgery, an on demand tool for clinician to verify proper functionality of the system, adjust one or more sensing parameters, or adjust one or more stimulation parameters related to therapy.

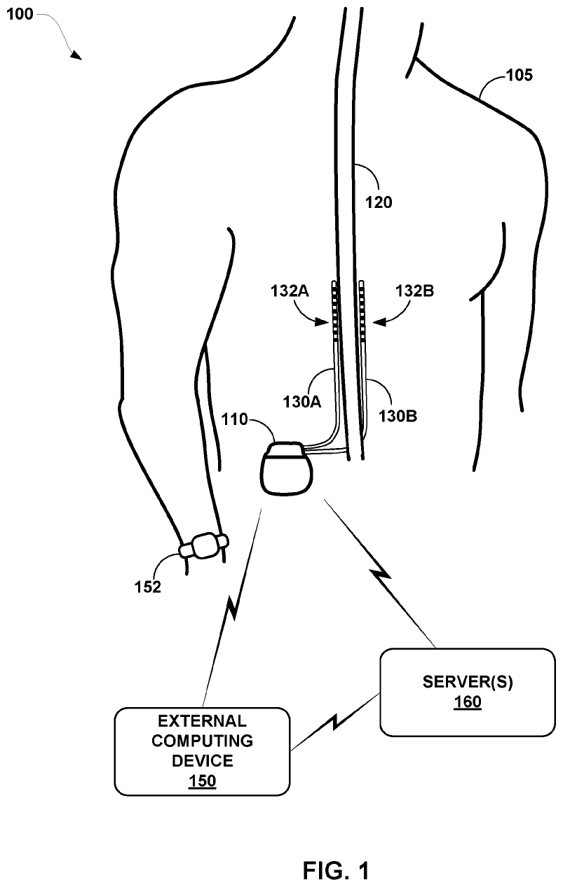

FIG. 1 is a conceptual diagram illustrating an example system 100 that includes an implantable medical device (IMD) 110 configured to deliver spinal cord stimulation (SCS) therapy and an external computing device 150, in accordance with one or more techniques of this disclosure. Although description may focus on implantable electrical stimulators (e.g., neurostimulators) for purposes of illustration, the techniques described in this disclosure are generally applicable to a variety of medical devices including external devices and IMDs. In other words, the disclosure will refer to an implantable SCS system for purposes of illustration, but without limitation as to other types of medical devices or other therapeutic applications of medical devices such as bioelectrical signal monitoring and therapy, peripheral nerve stimulation, deep brain stimulation (DBS) and similar devices.

As shown in FIG. 1, system 100 includes an IMD 110, leads 130A and 130B, and external computing device 150 shown in conjunction with a patient 105, who is ordinarily a human patient. In the example of FIG. 1, IMD 110 is an implantable electrical stimulator that is configured to generate and deliver electrical stimulation therapy to patient 105 via one or more electrodes 132A and 132B of leads 130A and/or 130B (collectively, “leads 130”), e.g., for relief of chronic pain or other symptoms. In other examples, IMD 110 may be coupled to a single lead carrying multiple electrodes, to more than two leads each carrying multiple electrodes, a system without leads and where the electrodes are disposed on the housing of the IMD, or any combination of such systems.

IMD 110 may be a chronic electrical stimulator that remains implanted within patient 105 for weeks, months, or even years. In other examples, IMD 110 may be a temporary, or trial, stimulator used to screen or evaluate the efficacy of electrical stimulation for chronic therapy. In one example, IMD 110 is implanted within patient 105, while in another example, IMD 110 is an external device coupled to percutaneously implanted leads. In some examples, IMD 110 uses one or more leads, while in other examples, IMD 110 is leadless. In other examples, IMD 110 may be located to deliver sacral neuromodulation (SNM), deep brain stimulation (DBS), and peripheral nerve stimulation (PNS), including tibial nerve stimulation (TNS), not shown in FIG. 1.

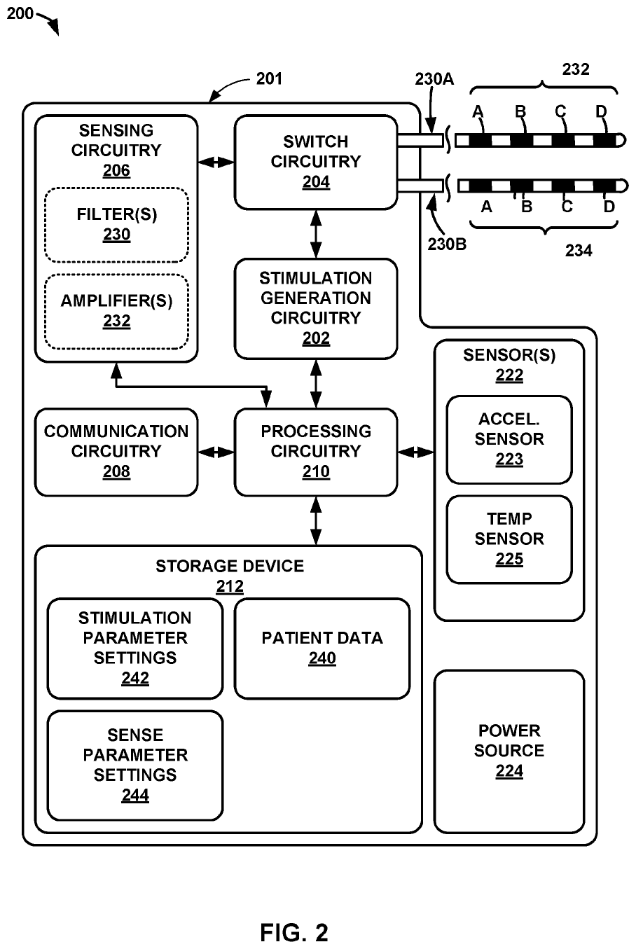

IMD 110 may be constructed of any polymer, metal, or composite material sufficient to house the components of IMD 110 (e.g., components illustrated in FIG. 2A) within patient 105. In this example, IMD 110 may be constructed with a biocompatible housing, such as titanium or stainless steel, or a polymeric material such as silicone, polyurethane, or a liquid crystal polymer, and surgically implanted at a site in patient 105 near the pelvis, abdomen, or buttocks. In other examples, IMD 110 may be implanted within other suitable sites within patient 105, which may depend, for example, on the target site within patient 105 for the delivery of electrical stimulation therapy. The outer housing of IMD 110 may be configured to provide a hermetic seal for components, such as a rechargeable or non-rechargeable power source. In addition, in some examples, the outer housing of IMD 110 is selected from a material that facilitates receiving energy to charge the rechargeable power source, if so equipped.

IMD 110 may deliver electrical stimulation energy, which may be constant current or constant voltage-based pulses, for example, to one or more target tissue sites of patient 105 via one or more electrodes of implantable leads 130. In the example of FIG. 1, leads 130 carry electrodes that are placed adjacent to the target tissue of spinal cord 120. One or more of the electrodes 132A or 132B (collectively electrodes 132) may be disposed at a distal tip of a lead 130 and/or at other positions at intermediate points along the lead. Leads 130 may be implanted and coupled to IMD 110. Electrodes 132 may transfer electrical stimulation generated by an electrical stimulation generator (e.g., stimulation generation circuitry) in IMD 110 to tissue of patient 105.

Electrodes 132 may also sense bioelectrical signals at the electrode-tissue interface. Some examples of bioelectrical signals may include local field potentials (LFP), evoked response style sensing (e.g., electrically evoked compound action potential (EECAP), evoked resonant neural activity (ERNA), evoked compound action potential (ECAP), electromyogram (EMG), cardiac activity, patient respiration, impedance, and similar bioelectrical signals. An ECAP is a synchronous firing of a population of neurons which occurs in response to the application of a stimulus including, in some cases, an electrical stimulus by a medical device. The ECAP may be detectable as being a separate event from the stimulus itself, and the ECAP may reveal characteristics of the effect of the stimulus on the nerve fibers. In some examples, sensing circuitry of IMD 110 may also receive direct spectral content of heart rate and respiration for patient 105 from the electrodes 132.

In some examples, system 100 may be configured to output an audio representation of the measured physiological signals including elements of the physiological signals such as bioelectrical signals, as well as other signals captured by sensors of IMD 110. For example, processing circuitry of system 100 may receive a signal from a temperature sensor, e.g., located on IMD 110 and output a sound that changes in pitch, intensity or some other audio feature as the temperature changes. In other examples, system 100 may use audio to represent the multi-component nature of the spinal signal to a clinician to inform clinical decisions related to configuring the system to the patient. In some examples, the captured bioelectrical, or other sensed signal, may contain stimulation artifact, LFP, ECAP, EMG activity, ECG, breathing signal, and noise. When configuring IMD 110, e.g., after implant surgery, challenges for the caregivers may include to quickly identify the nature of the observed spinal signal, or other sensed signal, for the clinician and to determine whether the system is operating correctly, and to select parameters and operating settings specific to the anatomy of patient 105 and the condition and symptoms of patient 105. For example, in a closed-loop ECAP system, it may be desirable to determine if there ECAP signal is present, and if there are additional signals (such as EMG) which may interfere with ECAP acquisition. The clinician may use this information to optimize the stimulation and the acquisition parameters of the system.

In addition to presenting a representation of measured sensed signals, for example on a display screen of external computing device 150, System 100 of this disclosure may play distinct sounds representing, for example, the presence of an ECAP signal and EMG noise during ECAP optimization, with volume or other characteristic representing strength of the respective signal. In some examples, system 100 may be configured to mask, filter or otherwise not present a sound when the signal detected is from the stimulation artifact, system noise, or other unwanted or distracting signals. In some examples, multiple signals may be combined into a single soundtrack, e.g. by having temporally and spectrally different sounds. In other examples the multiple signals may be presented in different soundtracks, e.g. stereo sound with different sounds coming from either real or virtual locations, such as left or right speaker/headphone or other virtual audio.

In some examples, a continuous sound that provides an indication of sensed signals, for example, when setting up closed loop operation of for IMD 110 may allow clinician to easily assess how well ECAP signals are controlled with the closed loop features during aggressors. In this disclosure, “aggressors” may include physical movements that may impact sensed bioelectrical signals, such as during a cough, sneeze, torso twisting or arching and other aggressors. The presence of EMG activity during aggressors (once identified by clinician) may lead the clinician to select different settings of closed-loop system. In some examples, the measured bioelectrical signal may include cardiac modulation of the ECAP, which may differ from measurements of LFP near the electrodes.

In some examples, the clinician may select and adjust the audio presentation of sensed signals in this disclosure, such as via a user interface for a computing device of system 100, e.g., a user interface for external computing device 150, 152 or remotely using a user interface connected to servers 160. For example, on demand, ECG signal and breathing signal derived from leads may be isolated and played for the clinician. The clinician may then determine whether the two signals are sufficiently clean, and in response to setting sensing and/or filtering parameters for the signals, the clinician may enable tracking of the ECG and breathing in IMD 110.

In some examples, system 100 may also include demonstration and training modes to train system users, including patients, clinicians, and other caregivers, on the audio presentations of the system. In some examples, demonstration systems may simulate different intended behaviors of the system, e.g. open-loop or closed-loop operation of the system and output the associated audio. In other examples, the system may create demonstrations of various abnormal scenario: e.g. presence of EMG signal to train clinicians as well as create demonstrations of ECG and breathing signals, both in ideal and in non-ideal conditions. In some examples, the signals may be created by simulating system 100 as well as patient response with a computer model.

As described above, sensing circuitry of IMD 110 may receive bioelectrical signals through electrodes 132 attached to leads 130. In the example of FIG. 1, leads 130 may each be a single lead. In other examples, leads 130 may include a lead extension or other segments that may aid in implantation or positioning of lead 130. In some other examples, IMD 110 may be a leadless stimulator with one or more arrays of electrodes arranged on a housing of the stimulator rather than leads that extend from the housing. In addition, in some other examples, system 100 may include one lead or more than two leads, each coupled to IMD 110 and directed to similar or different target tissue sites.

Electrodes 132 of leads 130 may be electrode pads on a paddle lead, such as a 5-6-5 lead, circular (e.g., ring) electrodes surrounding the body of the lead, conformable electrodes, cuff electrodes, segmented electrodes (e.g., electrodes disposed at different circumferential positions around the lead instead of a continuous ring electrode), any combination thereof (e.g., ring electrodes and segmented electrodes) or any other type of electrodes capable of forming unipolar, bipolar or multipolar electrode combinations for stimulation therapy or for sensing.

The deployment of electrodes via leads 130 is described for purposes of illustration, but arrays of electrodes may be deployed in different ways. For example, a housing associated with a leadless stimulator may carry arrays of electrodes, e.g., rows and/or columns (or other patterns), to which shifting operations may be applied. Such electrodes may be arranged as surface electrodes, ring electrodes, or protrusions. As a further alternative, electrode arrays may be formed by rows and/or columns of electrodes on one or more paddle leads. In some examples, electrode arrays include electrode segments, which are arranged at respective positions around a periphery of a lead, e.g., arranged in the form of one or more segmented rings around a circumference of a cylindrical lead. In other examples, one or more of leads 130 are linear leads having multiple ring electrodes along the axial length of the lead. In another example, the electrodes are segmented rings arranged in a linear fashion along the axial length of the lead and at the periphery of the lead. Ring electrodes arranged at different axial positions at the distal ends of lead 130 in FIG. 1 will be described for purposes of illustration of the techniques of this disclosure.

In some examples, leads 130 includes one or more other sensors configured to allow IMD 110 to monitor one or more other parameters of patient 105, such as patient activity, pressure, temperature, or other characteristics. The one or more sensors may be in addition to, or in place of, therapy delivery by lead 130. In other examples, other sensors, such as accelerometers, gyroscopes, or other movement sensors may be configured to sense movement of the patient. In some examples, the processing circuitry may automatically adjust, based on the physical state of the patient, at least one sensing parameter of the set of sensing parameters. In some examples, the processing circuitry may also, or alternatively, automatically adjust one or more stimulation parameters of the set of stimulation parameters, as well as other operating parameters based on the physical state of patient 105.

Some examples of sensing parameters that the processing circuity may adjust include the spacing between the sensing electrodes, and/or the orientation (e.g., the electrode combination) of at least one sensing electrode. For example, IMD 110 may change which electrodes are used as sensing electrodes to change the proximity of the sensing electrodes to the target tissue. In some examples, IMD 100 may change the orientation (e.g., which segment(s) of a segmented electrode or other electrodes are used to sense) by automatically searching the plurality of electrodes for a sensing electrode combination to determine which electrodes may be used as sensing electrodes e.g., to minimize the impact (e.g., amplitude) of a stimulation artifact or to maximize the signal strength of the sensed evoked response signal. In other examples, processing circuity of IMD 110 may also change a sensing channel, e.g., from LFP to ECAP or some other sensing channel adjustment based on the determined physical state.

Other sensing parameters of the set of sensing parameters, may include how the processing circuitry determines the characteristic value of the sensed evoked response signal. Other potential techniques for determining the characteristic value may include latency of a peak relative to a stimulation artifact, latency between peaks, the number of peaks, the slope of the artifact, decay constant of the artifact, polarity of the artifact, and/or other morphology of the artifact or morphology of the sensed evoked response signal.

In some examples, ECAP latency (generally ECAP type) may be indicative of lateral-to-medial placement of the stimulating lead. The audio presentation features of this may represent signal latency such as by generating a specific signal that indicates latency. Signal latency, e.g., between two ECAP signals, may be indistinguishable to the human ear in an audio representation. The ECAP with higher latency may just have a phase delay, but a similar frequency and so may sound the same. Processing circuitry of system 100 may transform the audio representation to distinguish the measured signals. For example, processing circuitry may change frequency or pitch of the signal with higher latency, modify its timbre or temporal envelope, or convert that signal to a different sound.

During placement surgery, system 100 may generate specific signal indicative of ECAP latency/ECAP type to assist the clinician in locating the electrodes in relation to the target tissue. The audio presentation of this disclosure may have the advantage of allowing the clinician to focus on placement without having to swap attention back and forth to a visual display of the ECAP signal or a fluoroscopic image as part of the implant procedure to adjust the desired placement. During post-operative programming optimization, in some examples, system 100 may use a virtual cathode to optimize lateral to medial placement of the center of the stimulation relative to physiological baseline. While clinician is changing the virtual cathode, the processing circuitry may play the audio to indicate the ECAP type. Other advantages of the audio output features of this disclosure may also include the difference between to see on the display screen that a desired measured signal is partially effaced or confounded by something else, however, hearing the difference in the measured signal, e.g., based on changing to different electrode combinations, or changing other parameters may be easier and more intuitive for the clinician and deliver better outcomes for patient 105.

The set of stimulation parameters of a therapy stimulation program that defines the stimulation signal of electrical stimulation therapy by IMD 110 through electrodes 132 of leads 130 may include information identifying which electrodes have been selected for delivery of stimulation according to a stimulation program, the polarities of the selected electrodes, i.e., the electrode combination for the program, voltage or current amplitude, pulse rate (e.g., pulse frequency), pulse width, pulse shape of stimulation delivered by the electrodes, a number of interleaved pulses, passive recharge settings, etc. These stimulation parameter values that make up the stimulation parameter set that defines the stimulation signal may be predetermined parameter values defined by a user and/or automatically determined by system 100 based on one or more factors or user input.

IMD 110 may configured to deliver electrical stimulation therapy to patient 105 via selected combinations of electrodes carried by one or both of leads 130, alone or in combination with an electrode carried by or defined by an outer housing of IMD 110. The target tissue for the electrical stimulation therapy may be any tissue affected by electrical stimulation, which may be in the form of electrical stimulation pulses or continuous waveforms. In some examples, the target tissue includes nerves, smooth muscle, or skeletal muscle of the anatomy of patient 105. In the example illustrated by FIG. 1, the target tissue is tissue proximate spinal cord 120, such as within an intrathecal space or epidural space of spinal cord 120, or, in some examples, adjacent nerves that branch off spinal cord 120. Leads 130 may be introduced into spinal cord 120 in via any suitable region, such as the thoracic, cervical, or lumbar regions. Stimulation of spinal cord 120 may, for example, prevent pain signals from traveling through spinal cord 120 and to the brain of patient 105. Patient 105 may perceive the interruption of pain signals as a reduction in pain and, therefore, efficacious therapy results. In other examples, stimulation of spinal cord 120 may produce paresthesia which may be reduce the perception of pain by patient 105, and thus, provide efficacious therapy results.

A user, such as a clinician or patient 105, may interact with a user interface of an external computing device 150 to program IMD 110. Programming of IMD 110 may refer generally to the generation and transfer of commands, programs, or other information to control the operation of IMD 110. In this manner, IMD 110 may receive the transferred commands and programs from external computing device 150 to control stimulation, such as electrical stimulation therapy to develop the growth curve. For example, external computing device 150 may transmit therapy stimulation programs, evoked response stimulation programs, stimulation parameter adjustments, therapy stimulation program selections, evoked response program selections, user input, or other information to control the operation of IMD 110, e.g., by wireless communication or wired connection.

In some cases, external computing device 150 may be characterized as a physician or clinician programmer if it is primarily intended for use by a physician or clinician. In other cases, external computing device 150 may be characterized as a patient programmer if it is primarily intended for use by a patient. A patient programmer may be generally accessible to patient 105 and, in many cases, may be a portable device that may accompany patient 105 throughout the patient's daily routine. For example, a patient programmer may receive input from patient 105 when the patient wishes to terminate or change electrical stimulation therapy, when a patient perceives stimulation being delivered or when a patient terminates due to comfort level. In general, a physician or clinician programmer may support selection and generation of programs by a clinician for use by IMD 110, whereas a patient programmer may support adjustment and selection of such programs by a patient during ordinary use.

In other examples, external computing device 150 may include, or be part of, an external charging device that recharges a power source of IMD 110. In this manner, a user may program and charge IMD 110 using one device, or multiple devices. External computing device may be any device with processing circuitry configured to communicate with other computing devices and perform calculations and other processing tasks. Some examples of external computing device 150 include wearable device 152, such as a fitness tracker, a tablet or laptop computer, a mobile phone or similar computing device (not shown in FIG. 1).

As described herein, information may be transmitted between external computing device 150 and IMD 110. Therefore, IMD 110 and external computing device 150 may communicate via wireless communication using any techniques known in the art. Examples of communication techniques may include, for example, radiofrequency (RF) communication and inductive coupling, but other techniques are also contemplated. In some examples, external computing device 150 includes a communication head that may be placed proximate to the patient's body near the IMD 110 implant site to improve the quality or security of communication, and/or power transfer, between IMD 110 and external computing device 150. Communication between external computing device 150 and IMD 110 may occur during power transmission or separate from power transmission.

In some examples, IMD 110 may also detect evoked response signals from the stimulation signal delivered to patient 105 for the purpose of modifying therapy delivered to the patient. During delivery of control stimulation pulses defined by one or more evoked response test stimulation programs, IMD 110, via two or more electrodes interposed on leads 130, senses electrical potentials of tissue of the spinal cord 120 of patient 105 to measure the electrical activity of the tissue. IMD 110 senses evoked response from the target tissue of patient 105, e.g., with sensing electrodes on one or more leads 130 and associated sensing circuitry. In some examples, IMD 110 receives a signal indicative of the evoked response from one or more sensors, e.g., one or more electrodes and circuitry, internal or external to patient 105. Such an example signal may include a signal indicating an evoked response of the tissue of patient 105.

Once the target evoked response characteristic values are set, and in addition to determining the physical state of patient 105, the example techniques allow for closed loop automatic adjustment of parameter values of the set of parameter values that define the stimulation signal to maintain consistent volume of neural activation and consistent perception of therapy for the patient. The ability to change the stimulation parameter values may also allow the therapy to have long term efficacy, with the ability to keep the intensity of the stimulation (e.g., as indicated by the evoked response) consistent by comparing the measured evoked response values to the target evoked response characteristic value. In addition, or alternatively, to maintaining stimulation intensity, IMD 110 may monitor the characteristic values of the evoked response signals to limit one or more parameter values that define the stimulation signal. IMD 110 may perform these changes without intervention by a physician or patient 105. In this manner, IMD 110 may deliver closed loop stimulation therapy.

Moreover, an IMD and/or electrodes used for stimulation and sensing may migrate within the patient, or the patient may change postures placing more or less pressure on areas containing the IMD and/or lead(s) thereby causing the electrodes, e.g., attached to the housing of IMD 110 or electrodes 132 on leads 130, to move closer or farther from the target tissue than at other times. Additionally, a patient disease state may change over time. Therefore, it may be desirable to dynamically and automatically change parameters, such as sensing parameters and/or stimulation parameters to more accurately identify the evoked response and thereby configure the electrical stimulation in a closed loop manner to deliver more efficacious therapy.

In some examples, IMD 110 may include artifact rejection circuitry which may include one or more filters to remove or reduce the impact of any artifact in the sensed evoked response signal. For example, IMD 110 may adapt stimulation parameters when an artifact in the sensed evoked response signal temporally shifts within a sensing window. IMD 110 may adapt stimulation parameters based on the magnitude (e.g., amplitude or area under the curve) of the artifact, the slope of the artifact, decay constant of the artifact, polarity of the artifact, and/or other morphology of the artifact. IMD 110 may adjust filtering parameters, such as frequency levels or filtering coefficients, based on the magnitude (e.g., amplitude or area under the curve) of the artifact, the slope of the artifact, decay constant of the artifact, polarity of the artifact, and/or other morphology of the artifact. IMD 110 may also adjust filtering parameters, or other artifact rejection parameters based on the determined physical state of patient 105.

Operating parameters may also include the operation of amplification circuitry of IMD 110. IMD 110 may include amplification circuitry to amplify the sensed evoked response signal and other bioelectrical signals. IMD 110 may determine a window, having a time duration, for amplification of the sensed evoked response signal and determine blanking parameters. IMD 110 may weight temporal data within the window and/or may weight calculated features (e.g., within the morphology of the temporal data) of the temporal data within the window. In some examples, the weighting may be different for different portions of the window. For example, the weighting may be configured to emphasize the evoked response or emphasize the artifact. Processing circuitry of IMD 110 may automatically adjust the operation of the amplification circuitry based on the physical state of patient 105.

In other examples, IMD 110 may adjust a gain or other parameters of the amplification circuitry based on at least one of the calculated features or the weighted calculated features. Such calculated features may include the magnitude (e.g., amplitude or area under the curve) of the artifact, the slope of the artifact, decay constant of the artifact, polarity of the artifact, and/or other morphology of the artifact or of the sensed evoked response signal.

Other examples of sensing parameters may include a change in the polarity of the stimulation electrodes from one polarity to the opposite polarity, back and forth, and average the resulting sensed evoked response signal. In some examples, IMD 110 may also provide a masker pulse which may delivered prior to the stimulation pulse such that the neural response to the stimulation pulse is masked due to neurons being in the refractory period due to their activation in response to the masker pulse. To facilitate this technique, the masker pulse may precede the stimulation pulse by either absolute or relative refractory period of the neural tissue (e.g., 0.3 to 5 msec). Because the neural response is masked, the recording due to pulse following the masked pulse may contain predominantly a stimulation artifact. This recording can then be used to estimate the artifact and subtract the artifact from subsequent stimulation pulses, to achieve a better estimate of underlying neural response.

In some examples, system 100 may include user controls to remove the artifact from the presented audio signal. For example, the artifact timing is known, so processing circuitry of system 100 may apply blanking to the signal to remove the artifact allow the clinician to focus on other portions of the multi-channel signal, e.g., EMG, breathing, and ECAPs. In some examples, system 100 may subtract a model of the stimulation artifact from the recorded signal to remove residual noise, or apply a high pass (HP) filter. In other examples, after blanking or otherwise removing the artifact, or other undesired portions of the measured signal, system 100 may apply band-pass filter to ensure smooth transitions. In other examples, system 100 may also fill transitions with previous samples, taper the signal before and after blank portions to avoid abrupt transitions or otherwise process the signal so that the audio presentation provides useful feedback to the user.

In the example of FIG. 1, IMD 110 is described as performing a plurality of processing and computing functions. However, external computing device 150, servers 160 or other processing circuitry of system 100 instead may perform one, several, or all of these functions. In this alternative example, IMD 110 functions to relay sensed signals to external computing device 150 for analysis, and external computing device 150 transmits instructions to IMD 110 to adjust the one or more stimulation parameters defining the electrical stimulation therapy based on analysis of the sensed signals. In some examples, processing circuitry of IMD 110, or other processing circuitry of system 100 may receive information from other devices of system 100, such as from wearable device 152 to determine the physical state of patient 105.

FIG. 2 is a block diagram illustrating an example combination of components of IMD 200, in accordance with one or more techniques of this disclosure. IMD 200 may be an example of IMD 110 of FIG. 1. In the example shown in FIG. 2, IMD 200 includes stimulation generation circuitry 202, switch circuitry 204, sensing circuitry 206, communication circuitry 208, processing circuitry 210, storage device 212, sensor(s) 222, and power source 224.

In the example shown in FIG. 2, storage device 212 may store patient data 240, stimulation parameter settings 242, sense parameter settings 244 and various thresholds discussed in this disclosure in separate memories within storage device 212 or separate areas within storage device 212. Patient data 240 may include parameter values, target characteristic values, recorded sensed signals, or other information specific to the patient. In some examples, stimulation parameter settings 242 may include stimulation parameter values for respective different stimulation programs selectable by the clinician or patient for therapy. In this manner, each stored therapy stimulation program, or set of stimulation parameter values, of stimulation parameter settings 242 defines values for a set of electrical stimulation parameters (e.g., a stimulation parameter set), such as a stimulation electrode combination, electrode polarity, current or voltage amplitude, pulse width, pulse rate, pulse shape, duty cycle, number of interleaved pulses, passive recharge settings, etc. Storage device 212 may also store sense parameter settings 244 that defines values for a set of electrical stimulation parameters configured to elicit a detectable evoked response signal, such as a stimulation electrode combination, electrode polarity, current or voltage amplitude, pulse width, pulse rate, and pulse shape, as described above in relation to FIG. 1.

Accordingly, in some examples, stimulation generation circuitry 202 generates electrical stimulation signals in accordance with the set of electrical stimulation parameters noted above. Other ranges of stimulation parameter values may also be useful and may depend on the target stimulation site within patient 105. Stimulation signals may be of any form, such as continuous-time signals (e.g., sine waves) or the like.

Switch circuitry 204 may include one or more switch arrays, one or more multiplexers, one or more switches (e.g., a switch matrix or other collection of switches), or other electrical circuitry configured to direct stimulation signals from stimulation generation circuitry 202 to one or more of electrodes 232, 234, or directed sensed signals from one or more of electrodes 232, 234 to sensing circuitry 206. In other examples, stimulation generation circuitry 202 and/or sensing circuitry 206 may include sensing circuitry to direct signals to and/or from one or more of electrodes 232, 234, which may or may not also include switch circuitry 204. Though shown as attached to leads 230, any of electrodes 232 or 234 may be part of housing 201 or attached to housing 201 of IMD 200.

Sensing circuitry 206 is configured to monitor signals from any combination of electrodes 232, 234 according to the set of sensing parameters 244. In some examples, sensing circuitry 206 includes one or more amplifiers, filters, and analog-to-digital converters. For example, amplifier(s) 232 of sensing circuitry 206 may amplify a sensed evoked response signal and/or filter(s) 230 of sensing circuitry 206 may filter a sensed evoked response signal which may be used to remove or reduce the impact of artifacts on a sensed evoked response signal. Sensing circuitry 206 may be used to sense physiological signals, such as evoked response signals and the sensing artifact. In some examples, sensing circuitry 206 detects evoked response from a particular combination of electrodes 232, 234. In some cases, the particular combination of electrodes for sensing evoked response includes different electrodes than a set of electrodes 232, 234 used to deliver stimulation pulses. Alternatively, in other cases, the particular combination of electrodes used for sensing evoked response includes at least one of the same electrodes as a set of electrodes used to deliver stimulation pulses to patient 105. Sensing circuitry 206 may provide signals to an analog-to-digital converter (not shown in FIG. 2), for conversion into a digital signal for processing, analysis, storage, or output by processing circuitry 210. As described above, and in relation to FIG. 1, processing circuitry may automatically change a sensing parameter of the set of sensing parameters based on the determined patient state.

Communication circuitry 208 supports wireless communication between IMD 200 and an external programmer (not shown in FIG. 2) or another computing device e.g., as shown in system 100 of FIG. 1. Processing circuitry 210 of IMD 200 may receive, as updates to programs, values for various stimulation parameters such as amplitude and electrode combination, from the external programmer via communication circuitry 208. Processing circuitry 210 may store updates to the stimulation parameter settings 242 or any other data in storage device 212. Communication circuitry 208 in IMD 200, as well as communication circuitry in other devices and systems described herein, such as the external computing device, may accomplish communication by radiofrequency (RF) communication techniques. In addition, communication circuitry 208 may communicate with an external medical device programmer (not shown in FIG. 2) via proximal inductive interaction of IMD 200 with an external computing device.

In some examples, communication circuitry 208 may transmit sensed signals to external computing devices, such as computing device 150 and/or servers 160 described above in relation to FIG. 1. The sensed signals may be real-time or near real-time or may be signals stored at patient data 240. In some examples, processing circuitry 210 may perform some of the audio signal processing described in this disclosure, e.g., filtering, amplification, modifying the received signal to present the signal in an audible range, and combining different measured signals, like LFP and ECAP into an audio feedback signal. In other examples, other processing circuitry, e.g., of system 100, may perform the audio signal processing and outputting the audio signals to the user.

In some examples, IMD 200 may lack the data bandwidth to stream continuous ECAP channel samples, or other bioelectrical and sensed signals. In some examples, processing circuitry 210 may control communication circuitry 208 to only stream during a portion of the communication bandwidth. As one example, IMD 200 may only stream a few milliseconds (ms) of a transmit window, e.g., stream up two milliseconds of every twenty milliseconds, and may be centered around a window where sensing circuitry 206 expects an ECAP. In some examples, to output a continuous audio feedback of ECAP signals, the sensed ECAP transformed to an audio signal may operate with some dead space. For example, only transmit the ECAP for a small percent of the time, e.g., 10% in the example above. In this situation a system including IMD 200, e.g., system 100 of FIG. 1, may apply a window function, e.g. Hann, Blackman, or Gaussian windows, to the sensed ECAP waveform snippet so the ends are tapered to zero into and out of the dead space. In other examples, processing circuitry of the system of this disclosure may fill that space with repeated values or artificially generated noise. In other examples, the ECAP window function may also operate to optimize the spectral design, for example to limit spectral spread at the transition between ECAPS. In addition, the system may be configured to operate by streaming a reduced number of ECAP windows; e.g. every second measured ECAP will be streamed. In that case, for generating audio demos, the missing ECAPS can be generated by interpolating between acquired waveforms, or other techniques. Finally, instead of streaming the waveform, waveform may be compressed by various techniques, e.g. principal component analysis. In that case, the system would decompress the waveforms prior to generating audio.

In some examples, IMD 200 may lack the data bandwidth to stream continuous ECAP channel samples, or other bioelectrical and sensed signals. Audio signal processing may be done on the implanted device and a compressed audio representation of the post-processed audio, e.g. mp3 compression format, may be streamed within the available communication bandwidth to an external computing device of the system, e.g., any external computing device described above in relation to FIG. 1.

The external computing device may be one example of external computing devices 150, 152 or servers 160 of FIG. 1. Accordingly, communication circuitry 208 may send information to the external computing device on a continuous basis, at periodic intervals, or upon request from the external computing device. As described above in relation to FIG. 1, processing circuitry of system 100 may receive information from IMD 200 and may perform any of the analysis or signal processing described herein. If external to IMD 200, the external processing circuitry may communicate with processing circuitry 210 to control delivery of the electrical stimulation according to the adjusted value of the at least one stimulation parameter. Similarly, processing circuitry of system 100, such as processing circuity of IMD 110, may automatically adjust at least one sensing parameter of the set of sensing parameters including artifact cancellation parameters, based on determining a change in patient physical state. In other examples, a user may manipulate controls on a user interface of an external computing device to adjust the sensing parameters used by sensing circuitry 206 of IMD 200 in response to the feedback from the audio signal output, as described above in relation to FIG. 1.

Processing circuitry 210 may include any one or more of a microprocessor, a controller, a digital signal processor (DSP), an application specific integrated circuit (ASIC), a field-programmable gate array (FPGA), discrete logic circuitry, or any other processing circuitry configured to provide the functions attributed to processing circuitry 210 herein may be embodied as firmware, hardware, software, or any combination thereof. Processing circuitry 210 controls stimulation generation circuitry 202 to generate stimulation signals according to stimulation parameter settings 242 and any other instructions stored in storage device 212 to apply stimulation parameter values specified by one or more of programs, such as electrode combination, electrode polarity, amplitude, pulse width, pulse rate, pulse shape, number of interleaved pulses, passive recharge settings, etc., of each of the stimulation signals.

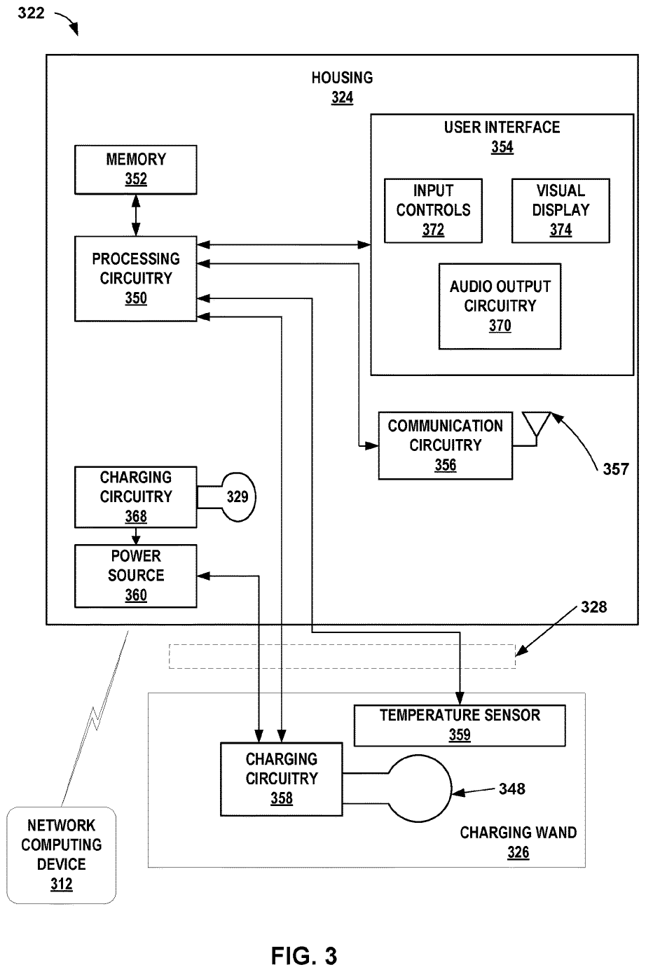

In the example shown in FIG. 2, lead 230A is shown having a set of electrodes 232 that includes electrodes 232A-232D, and lead 230B is shown having a set of electrodes 234 includes electrodes 234A-234D. However, lead 230A and lead 230 B may have any number of electrodes, such as 8 electrodes or 16 electrodes. In other examples, a single lead may be coupled to IMD 200 which may include any number of electrodes, such as include 8 electrodes or 16 electrodes along a single axial length of the lead. In some examples, one or more leads may include electrodes as shown in FIGS. 3A-3E. In other examples, as described above in relation to FIG. 1, IMD 200 may include electrodes on the housing of IMD 200 and in some examples be configured as a leadless device (not shown in FIG. 2)

Processing circuitry 210 also controls stimulation generation circuitry 202 to generate and apply the stimulation signals to selected combinations of electrodes 232, 234. In some examples, stimulation generation circuitry 202 includes a switch circuit (instead of, or in addition to, switch circuitry 204) that may couple stimulation signals to selected conductors within leads 230, which, in turn, deliver the stimulation signals across selected electrodes 232, 234. Such a switch circuit may be a switch array, switch matrix, multiplexer, or any other type of switching circuit configured to selectively couple stimulation energy to selected electrodes 232, 234 and to selectively sense bioelectrical neural signals of a spinal cord of the patient (not shown in FIG. 2) with selected electrodes 232, 234. As described above in relation to FIG. 1, electrodes 232 and 234 may also be configured to sense other bioelectrical signals.

In other examples, however, stimulation generation circuitry 202 does not include a switch circuit and switch circuitry 204 does not interface between stimulation generation circuitry 202 and electrodes 232, 234. In these examples, stimulation generation circuitry 202 includes a plurality of pairs of voltage sources, current sources, voltage sinks, or current sinks connected to each of electrodes 232, 234 such that each pair of electrodes has a unique signal circuit. In other words, in these examples, each of electrodes 232, 234 is independently controlled via its own signal circuit (e.g., via a combination of a regulated voltage source and sink or regulated current source and sink), as opposed to switching signals between electrodes 232, 234.

Electrodes 232, 234 on respective leads 230 may be constructed of a variety of different designs. For example, one or both of leads 230 may include one or more electrodes at each longitudinal location along the length of the lead, such as one electrode at different perimeter locations around the perimeter of the lead at each of the locations A, B, C, and D. In one example, the electrodes may be electrically coupled to stimulation generation circuitry 202, e.g., via switch circuitry 204 and/or switching circuitry of the stimulation generation circuitry 202, via respective wires that are straight or coiled within the housing of the lead and run to a connector at the proximal end of the lead. In another example, each of the electrodes of the lead may be electrodes deposited on a thin film. The thin film may include an electrically conductive trace for each electrode that runs the length of the thin film to a proximal end connector. The thin film may then be wrapped (e.g., a helical wrap) around an internal member to form the lead 230. These and other constructions may be used to create a lead with a complex electrode geometry.

Although sensing circuitry 206 is incorporated into a common housing with stimulation generation circuitry 202 and processing circuitry 210 in FIG. 2, in other examples, sensing circuitry 206 may be in a separate housing from IMD 200 and may communicate with processing circuitry 210 via wired or wireless communication techniques. In some examples, one or more of electrodes 232 and 234 are suitable for sensing the evoked responses. For instance, electrodes 232 and 234 may sense the voltage amplitude of a portion of the evoked response signals, where the sensed voltage amplitude, such as the voltage difference between features within the signal, is a characteristic the evoked response signal.

Processing circuitry 210 may automatically adapt stimulation parameters when an artifact in the sensed evoked response signal shifts in a sensing window. Processing circuitry 210 may determine that at least one of the artifact or the sensed evoked response signal is more and a difference threshold different than an expected evoked response signal and refrain from changing the stimulation parameters based on the sensed evoked response signal (e.g., temporarily stopping the use of closed-loop stimulation, in which case stimulation generation circuitry 202 may continue to deliver stimulation using the existing stimulation parameters).

Storage device 212 may be configured to store information within IMD 200 during operation. Storage device 212 may include a computer-readable storage medium or computer-readable storage device. In some examples, storage device 212 includes one or more of a short-term memory or a long-term memory. Storage device 212 may include, for example, random access memories (RAM), ferroelectric random access memories (FRAM), dynamic random access memories (DRAM), static random access memories (SRAM), magnetic discs, optical discs, flash memories, or forms of electrically programmable memories (EPROM) or electrically erasable and programmable memories (EEPROM). In some examples, storage device 212 is used to store data indicative of instructions for execution by processing circuitry 210. As discussed above, storage device 212 is configured to store patient data 240, stimulation parameter 214, and sense parameter settings 244.

In some examples, storage device 212 may store evoked response detection instructions which may include instructions on how processing circuitry 210 can adjust stimulation (e.g., stimulation parameter settings 242), the manner in which evoked responses are sensed, and/or the manner in which characteristic values are determined, in response to the determined characteristic values of evoked response signals or artifacts.

As described above in relation to FIG. 1, sensor(s) 222 may include one or more sensing elements that sense values of a respective patient parameter, such as posture state and activity state. Sensor(s) 222 may include one or more accelerometers, and gyroscopes, such as a micro electro-mechanical system (MEMS) based device. Other sensors may include optical sensors, chemical sensors, temperature sensors, pressure sensors, or any other types of sensors. Sensor(s) 222 may output patient parameter values that may be used as feedback to control delivery of therapy. For example, sensor(s) 222 may indicate physical state, including patient activity, and processing circuitry 210 may increase the frequency of control pulses and evoked response sensing in response to detecting increased patient activity.

In some examples, processing circuitry 210 may initiate control pulses and corresponding evoked response sensing in response to a signal from sensor(s) 222 indicating that patient activity has exceeded an activity threshold. Conversely, processing circuitry 210 may decrease the frequency of control pulses and evoked response sensing in response to detecting decreased patient activity. For example, in response to sensor(s) 222 no longer indicating that the sensed patient activity exceeds a threshold, processing circuitry 210 may suspend or stop delivery of control pulses and evoked response sensing. In this manner, processing circuitry 210 may dynamically deliver control pulses and sense evoked response signals based on patient activity to reduce power consumption of the system when the electrode-to-neuron distance is not likely to change and increase system response to evoked response changes when electrode-to-neuron distance is likely to change. IMD 200 may include additional sensors within the housing of IMD 200 and/or coupled via one of leads 230 or other leads. In addition, IMD 200 may receive sensor signals wirelessly from remote sensors via communication circuitry 208, for example. In some examples, one or more of these remote sensors may be external to patient (e.g., carried on the external surface of the skin, attached to clothing, or otherwise positioned external to patient 105, such as wearable device 152 shown in FIG. 1). In some examples, signals from sensor(s) 222 indicate the physical state including a position, body state, or posture (e.g., sleeping, awake, sitting, standing, or the like), and processing circuitry 210 may select target evoked response characteristic values according to the indicated physical state of the patient.

Power source 224 is configured to deliver operating power to the components of IMD 200. Power source 224 may include a battery and a power generation circuit to produce the operating power. In some examples, the battery is rechargeable to allow extended operation. In some examples, recharging is accomplished through proximal inductive interaction between an external charger and an inductive charging coil within IMD 200. Power source 224 may include any one or more of a plurality of different battery types, such as nickel cadmium batteries and lithium ion batteries.

FIG. 3 is a block diagram of an example an external computing device of FIG. 1. External computing device 322 in of FIG. 2 is an example of external computing device 150 described above in relation to FIG. 1. In some examples, external computing device 322 may be described as a hand-held device, in other examples, external computing device 322 may be a larger or a non-portable device. In addition, in other examples external computing device 322 may be included as part of an external programmer or include functionality of an external programmer. External computing device 322 may also be referred to as recharger 322, external charging device 322 or programmer 322 in this disclosure.

As shown in the example of FIG. 3, external computing device 322 includes two separate components. Housing 324 encloses components such as a processing circuitry 350, memory 352, user interface 354, communication circuitry 356, audio output circuitry 370 and power source 360. Charging head 326, also called charging wand 326, may include charging circuitry 358, temperature sensor 359, and coil 348. Housing 324 is electrically coupled to charging head 326 via charging cable 328. Housing 324 may also include charging circuitry 368 and coil 329, which is an example of coil 329 described above in relation to FIG. 1.

In some examples, separate charging wand 326 may facilitate positioning of coil 348 over coil 216 of IMD 210. In some examples, charging circuitry 368 and/or coil 329 may be integrated within housing 324 in other examples, as described above in relation to FIG. 1. In other examples, recharger 322 may not include charging wand 326. Memory 352 may store instructions that, when executed by processing circuitry 350, causes processing circuitry 350 and external computing device 322 to provide the functionality ascribed to external computing device 322 throughout this disclosure, and/or any equivalents thereof. Coil 348 and coil 329 may also be referred to as an antenna.

External computing device 322 may also include one or more temperature sensors, illustrated as temperature sensor 359, similar to temperature sensor 39 of FIG. 2. As shown in FIG. 3, temperature sensor 359 may be disposed within charging head 326. In other examples, one or more temperature sensors of temperature sensor 359 may be disposed within housing 324. For example, charging head 326 may include one or more temperature sensors positioned and configured to sense the temperature of coil 348 and/or a surface of the housing of charging head 326. In some examples, external computing device 322 may not include temperature sensor 359.

In general, external computing device 322 comprises any suitable arrangement of hardware, alone or in combination with software and/or firmware, to perform the techniques ascribed to external computing device 322, and processing circuitry 350, user interface 354, communication circuitry 356, and charging circuitry 358 of external computing device 322, and/or any equivalents thereof. In various examples, external computing device 322 may include one or more processors, such as one or more microprocessors, DSPs, ASICs, FPGAs, or any other equivalent integrated or discrete logic circuitry, as well as any combinations of such components. External computing device 322 also, in various examples, may include a memory 352, such as RAM, ROM, PROM, EPROM, EEPROM, flash memory, a hard disk, a CD-ROM, comprising executable instructions for causing the one or more processors to perform the actions attributed to them. Moreover, although processing circuitry 350, communication circuitry 356, charging circuitry 358, and temperature sensor 359 are described as separate modules, in some examples, processing circuitry 350, communication circuitry 356, charging circuitry 358, and/or temperature sensor 359 are functionally integrated. In some examples, processing circuitry 350, communication circuitry 356, charging circuitry 358, and/or temperature sensor 359 correspond to individual hardware units, such as ASICs, DSPs, FPGAs, or other hardware units.

Memory 352 may store instructions that, when executed by processing circuitry 350, cause processing circuitry 350 and external computing device 322 to provide the functionality ascribed to external computing device 322 throughout this disclosure, and/or any equivalents thereof. For example, memory 352 may include instructions that cause processing circuitry 350 to control the power level used to charge IMD 210 in response to the determined temperatures for the housing/external surface(s) of IMD 210, as communicated from IMD 210, or instructions for any other functionality. Memory 352 may include a record of selected power levels, sensed temperatures, determined temperatures, recorded bioelectrical signals or any other data. Processing circuitry 350 may, when requested, transmit any stored data in memory 352 to another computing device for review or further processing, such as to network computing device 312, as described above in relation to FIG. 2.

Network computing device 312 act as a server, such as a cloud based server, or a household server. In some examples network computing device 312 may be a tablet computer, laptop computer, desktop computer, mobile phone and so on. Network computing device 312 may include a user interface which may display outputs and accept inputs, such as the state of a patient's symptoms, as described above in relation to FIGS. 1 and 2. In this manner, a user interface of network computing device 312 may be described as being operatively coupled to processing circuitry 350 as well as to processing circuitry 230 depicted in FIG. 2.

Processing circuitry 350, may receive sensed signals from the medical device e.g., IMD 110 of FIG. 1. As described above, sensed signals may include evoked response signals such as ECAP, tissue impedance between electrodes, cardiac signals, breathing, EMG, e.g., caused by aggressors like movement, coughing, and similar functions that may generate EMG signals, as well as temperature, pressure, movement, and posture. In the example of the SCS system 100 depicted in FIG. 1, the spinal signal contains of information about the activity of the spinal cord and the patient. For example, the ECAPS response amplitude may indicated the effectiveness of stimulation and the impact of aggressors. ECAPS latency may indicate laterality of stimulation. An ECG may indicated cardiac health and emotional state. EMG activity may provide information about the activity of muscles near the sensing electrodes. Detected spinal traffic may show activity at remote sites in the spinal cord and measured LFP may indicate background activity of nearby tissue.

Processing circuitry 350, or other processing circuitry of the system, may output an audio signal representation of the sensed signals. Clinicians, patients, and other caregivers may can listen to audio representing the spinal signal without looking at the screen, such as visual display 374 and make clinical decisions. As described above in relation to FIGS. 1 and 2, some examples that may be provided by the audio output of this disclosure may include the presence of an ECAP, and how an ECAP changes with aggressors. Clinician may change sensing and/or therapy parameters and turn on or off closed loop response to determine the effectiveness of aggressor management on sensed signals. The audio output may present the latency of ECAP and hence how laterally the lead is placed relative to the target tissue. The audio representation of the lateral placement may be used to actively assist determine lead location during implant surgery. Processing circuitry 350 may cause audio output circuitry 370 to output an indication of the presence of a pain signal at a certain spinal level, for example as potential LFP peak. The spinal audio may be useful in demonstrations and training to explain and verify the medical technology features of the system. For example, the audio representation may be another way, e.g., along with a visual representation on visual display 374, to demonstrate spinal audio to clinicians to understand ECAPS and closed loop control. In some examples a user may correlate the processed audio signals of the sensed signals to the displayed indication of the sensed signals. The system may provide the audio spinal signal on demand as a tool for clinician to verify proper functionality of the system such as during patient follow-up visits, and to isolate unwanted noises. In some examples, the system may improve audio contrast by removing background and accentuating signal of interest, such as using a noise gate.

User interface 354 may receive user input with input controls 372. The input may be, for example, in the form of pressing a button on a keypad or selecting an icon from a touch screen. In some examples, the input may change programmed settings, start, or stop therapy, request starting or stopping a recharge session, a desired level of charging, or one or more statistics related to charging rechargeable power source 18 (e.g., the cumulative thermal dose). In this manner, user interface 354 may allow the user to view information related to the operation of the IMD.

The user input to input controls 372 of user interface 354 may also modify the audio output from audio output circuitry 370. User input may combine, or separate sensed signals to emphasize aspects of the multi-channel audio signal that the processing circuitry of this system may present to the user. In some examples a user may want to separate the ECAPS or LFP signals from any EMG noise. In other examples, a user may want to simultaneously output two or more signals, e.g., to compare the effects on each other, such as ECAPs on a right side headphone channel and cardiac signal or respiratory signal on a left side headphone channel, or any other combination of sensed signals, to provide virtual locations for the signals. Such stereo, or other multiple speaker output, may output different audio representations of signals at different virtual locations, e.g., left, right, center, high, low, and so on by utilizing virtual audio techniques available with two or more speakers. A similar technique may deliver spatially separated sounds (such as using different audio channels) for the signals received by different electrodes or electrode combinations to aid the clinician in tuning the sensing and evoked response parameters. For example, the ECAP signal sensed from a first electrode combination may be encoded in a first audio channel (such as a left audio channel) and the ECAP signal sensed from a second electrode combination may be encoded in a second audio channel. In other examples, the system may provide an audio output for any other sensed signals in one or more audio channels, such as the effect on static blood pressure changes, bladder filling, the impact of changes in medication on ECAP, LFP or other sensed signals.

In other examples, user interface 354 may receive an input from a user to define a stimulation pattern. User interface 354 may present selections and controls to the user and receive commands to define various parameters for each stimulation pattern, which in some examples may be customized patterns made up of approved stimulation constructs, e.g., biphasic waveforms, amplitude and/or pulse width ramping patterns, and so on. Processing circuitry 350 may execute programming instructions, e.g., stored at memory 352 and compile the defined stimulation pattern selected by the user into the instructions configured to be executed by the processing circuitry of a medical device, e.g., IMD 210. Processing circuitry 350 may output the instructions to the medical device via communication circuitry, e.g., communication circuitry 356, or in some examples, via inductive coupling using recharge circuitry 368 or 358.

Charging circuitry 358 may include one or more circuits that generate an electrical signal, and an electrical current, within primary coil 348. Charging circuitry 358 may generate an alternating current of specified amplitude and frequency in some examples. In other examples, charging circuitry 358 may generate a direct current. In any case, charging circuitry 358 may be capable of generating electrical signals, and subsequent magnetic fields, to transmit various levels of power to IMD 210. In this manner, charging circuitry 358 may be configured to charge rechargeable power source 18 of IMD 210 with the selected power level.

Power source 360 may deliver operating power to the components of external computing device 322. Power source 360 may also deliver the operating power to drive primary coil 348 during the charging process. Power source 360 may include a battery and a power generation circuit to produce the operating power. In some examples, a battery of power source 360 may be rechargeable to allow extended portable operation. In other examples, power source 360 may draw power from a wired voltage source such as a consumer or commercial power outlet.

Communication circuitry 356 supports wireless communication between IMD 210 and external computing device 322 under the control of processing circuitry 350. Communication circuitry 356 may also be configured to communicate with another computing device via wireless communication techniques, or direct communication through a wired connection. In some examples, communication circuitry 356 may be substantially similar to communication circuitry 236 of IMD 210 described herein, providing wireless communication via an RF or proximal inductive medium, e.g., using coil 348. In some examples, communication circuitry 356 may include an antenna 357, which may take on a variety of forms, such as an internal or external antenna. Although communication modules 356 and 36 may each include dedicated antennas for communications between these devices, communication modules 356 and 36 may instead, or additionally, be configured to utilize inductive coupling from coils 216 and 348 to transfer data.

In some examples, communication circuitry 356 may receive recorded or real-time sensed signals from an IMD, e.g., IMD 110 depicted in FIG. 1. In some examples, processing circuitry 350 may perform the audio signal processing described herein. In other examples, communication circuitry 356 may offload the received sensed signals to another computing device, such as servers 160 shown in FIG. 1. The second computing device may perform the audio processing steps, communication circuitry 356 may receive the audio signal and output the audio signal via audio output circuitry 370 of user interface 354. In other examples, the audio processing and audio output may be shared among any of the processing circuitry of the system of this disclosure, e.g., network computing device 312 implemented as a tablet computer, mobile phone or other device. Similarly, audio output circuitry 370 may be located on a device separate from processing circuitry 350.

Examples of local wireless communication techniques that may be employed to facilitate communication between external computing device 322 and IMD 210 include radio frequency and/or inductive communication according to any of a variety of standard or proprietary telemetry protocols, or according to other telemetry protocols such as the IEEE 802.11x or Bluetooth specification sets. In this manner, other external devices may be capable of communicating with external computing device 322 without needing to establish a secure wireless connection.

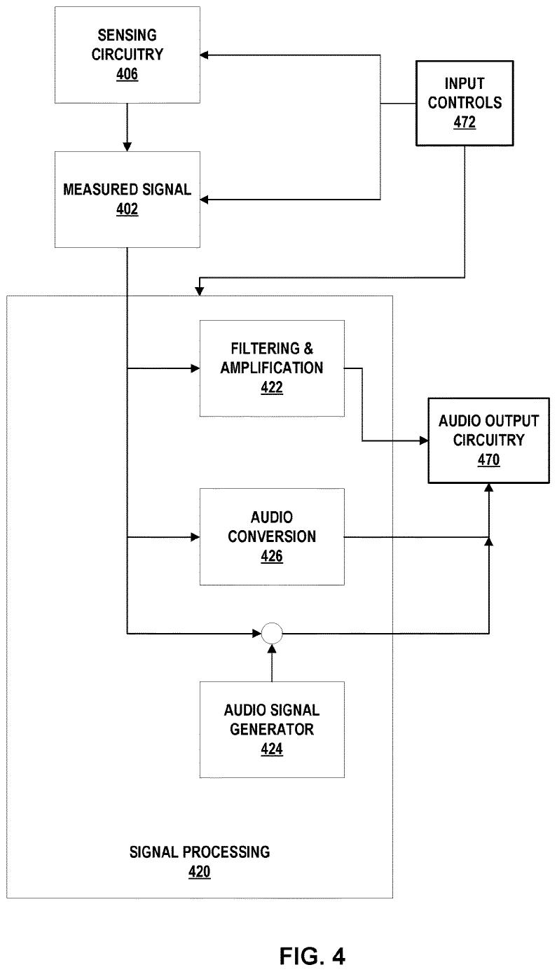

FIG. 4 is a block diagram illustrating an example of system components involved in outputting the audio signals described in this disclosure. The various components shown in FIG. 4, such as signal processing 420, may be all handled by processing circuitry of one computing device, or may be shared among two or more computing devices of a system, such as the systems described above in relation to FIGS. 1-3. In the example of FIG. 4, sensing circuitry 406 may be an example of sensing circuitry 206 and sensors 222 of FIG. 2, input controls 472 is an example of input controls 372 of user interface 354 of FIG. 3, and audio output circuitry 470 is an example of audio output circuitry 370 of FIG. 3.

In some examples, signal processing circuitry 420 may receive measured signal 402, which may include EMG, LFP, ECAP and other measured signals described above in relation to FIGS. 1-3. Measured signal 402 may be a real-time or previously recorded signal, e.g., retrieved from memory 352 of FIG. 3.

In some examples, the frequency of measured signal 402 in the audible range for a human ear. For example, an amplified ECAP signal may sound like a buzz. In response to user input from user controls 472, signal processing circuitry 420 may simply amplify the received measured signal 402 and audio output circuitry 470 may present the amplified signal to the user. Filtering and amplification circuitry 422 may also filter measured signal 402, e.g., to remove unwanted noise, and may apply different filters in response to user input. In some examples, filtering and amplification circuitry 422 may help remove or reduce the stimulation artifact from the audio signal. Filtering may include exponential filtering, digital filtering, adaptive filtering, time window blanking and other techniques. Exponential decay occurs naturally when a quantity is decaying at a rate which is proportional to how much is left. Undriven oscillations like the stimulation artifact may decay exponentially, therefore an exponential fit filter may be useful to remove or diminish the artifact.

In other examples, signal processing circuitry 420 may apply measured signal 402 to modulate the output of audio signal generator 424 to send an audio signal to audio output circuitry 470. In some examples, measured signal 402 may have a frequency component, but may not be in the audible range. Modulating the output of audio signal generator 424 may generate an audio signal that the user may hear to analyze the performance of the system. In other examples, measured signal 402 may be some other signal, e.g., without a frequency component, that by modulating the output from audio signal generator 424 may also generate a useful audio signal for the user. For example, signal processing circuitry 420 may emphasize latency in an ECAP by using modulation to frequency shift the ECAP signal. In other examples, audio signal generator 424 may fill in dead time, as described above, to output a continuous audio signal for the user. In other examples, signal processing circuitry 420 may help exaggerate differences between signals by moving the measured signals to frequency ranges, or other sound characteristics where the human ear is more sensitive to differences.

In other examples, signal processing circuitry 420 may use audio conversion circuitry 426 to generate an audio signal based on measured signal 402. In a manner similar to how a heart rate monitoring device generates a beep for each heart contraction and a flat tone in the event heart beats for a patient stop, audio conversion circuitry 426 may generate an audio signal to indicate the presence of measured signal 402, changes in measured signal 402 or other characteristics of measured signal 402. For example, audio conversion circuitry 426 may generate a series of tones to indicated detected spinal traffic showing activity at remote sites. Audio conversion circuitry 426 may change the tone frequency, timbre, repetition rate, and other characteristics as spinal activity changes. Similarly, audio conversion circuitry 426 may generate an audio output for any other measured signal 402 by using frequency scaling, or other techniques.

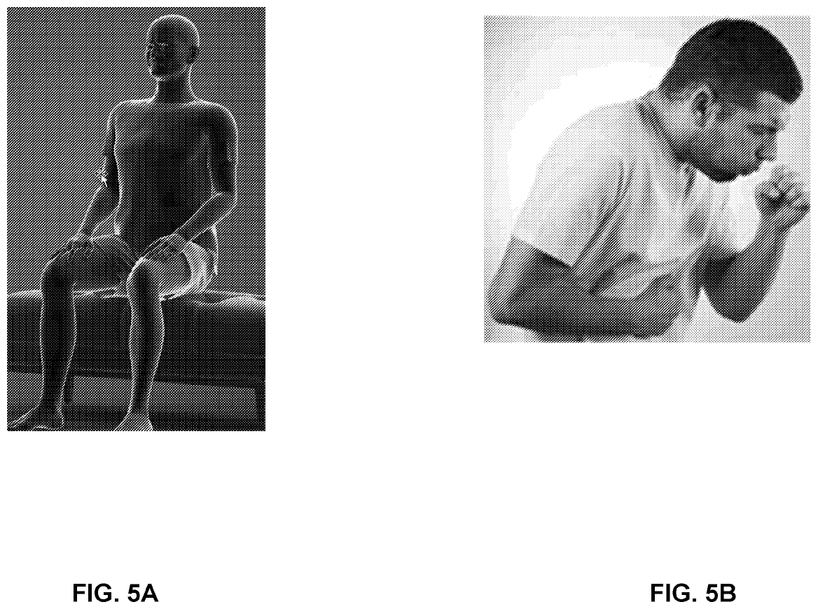

FIGS. 5A and 5B are conceptual diagrams illustrating aggressors that may impact a measured signal. As described above in relation to FIGS. changes in posture, movement and muscle contractions may affect one or more measured signals from a patient. Breathing and heartbeat may affect the measured ECAP. Similarly, movements, such as arching the back, shown in FIG. 5A, coughing, sneezing, and similar aggressors, shown in FIG. 5B may also affect measured signals, including the ECAP.

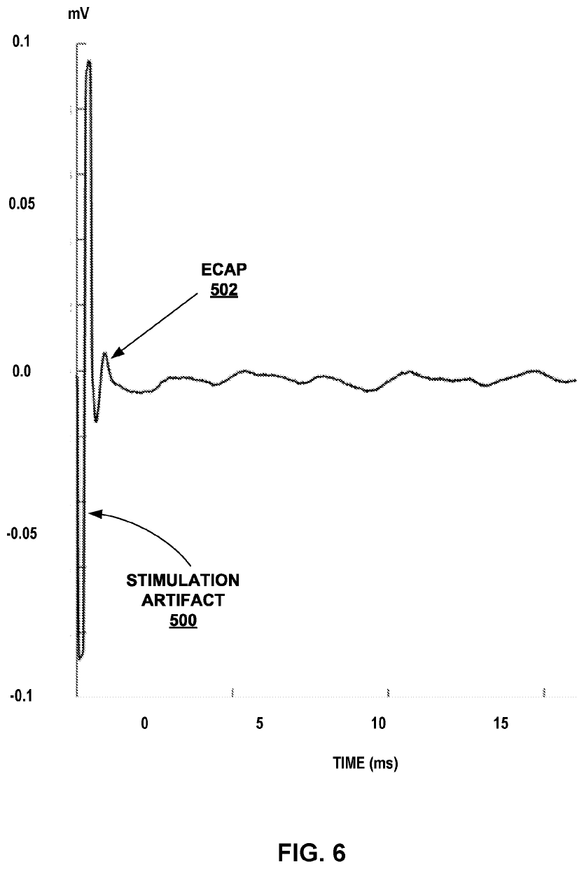

FIG. 6 is a timing diagram illustrating an example stimulation delivery and measured response according to one or more techniques of this disclosure. As described above in relation to FIGS. 1-3, an implantable medical device of this disclosure may deliver electrical stimulation therapy intended to capture a nerve, such as a spinal cord nerve depicted in FIG. 1. The device may deliver an electrical stimulation therapy event through one or more electrodes, e.g., electrodes 132 depicted in FIG. 1.

The delivered electrical stimulation therapy event may generate stimulation artifact 500, which the sensing circuitry of the device may perceive as a bioelectrical signal. In some examples, the device may deliver an electrical stimulation therapy event with enough energy, e.g., a control pulse, to elicit ECAP 502. In some examples, electrodes connected to the implantable medical device may be configured to sense any or all of the stimulation artifact, an EMG and ECAPs 502 and other bioelectrical signals. The specific timing may vary based on patient, location of the stimulation electrodes and location of the sensing device. As described above in relation to FIGS. 1-4, the medical device of this disclosure may output an audio signal representing any of the measured signals including artifact 500 and ECAP 502.

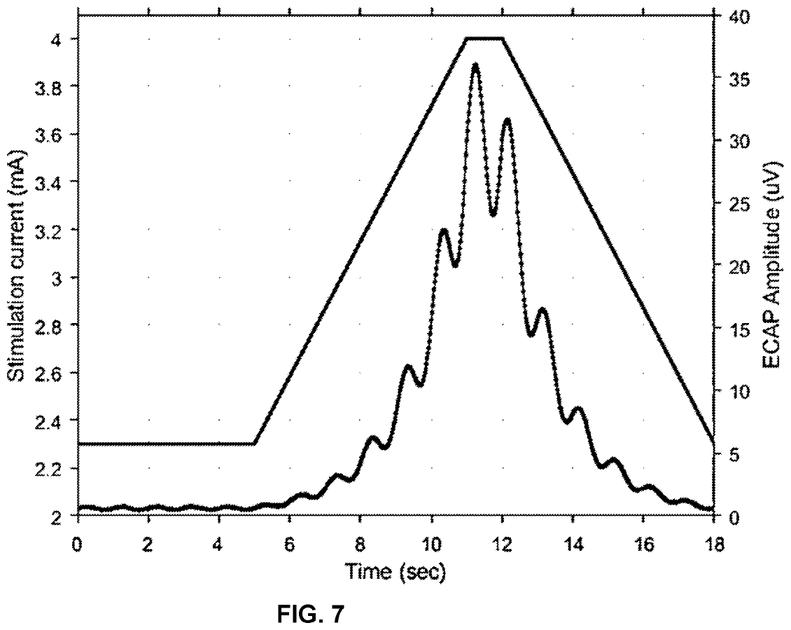

FIG. 7 is a time graph illustrating how changes in the stimulation current may affect the amplitude of an ECAP. As shown in the example of FIG. 7, increasing the stimulation amplitude (solid line) may cause a corresponding increase in the measured ECAP amplitude (dotted line). Similarly, as the stimulation amplitude decreases, the measure ECAP may also decrease.

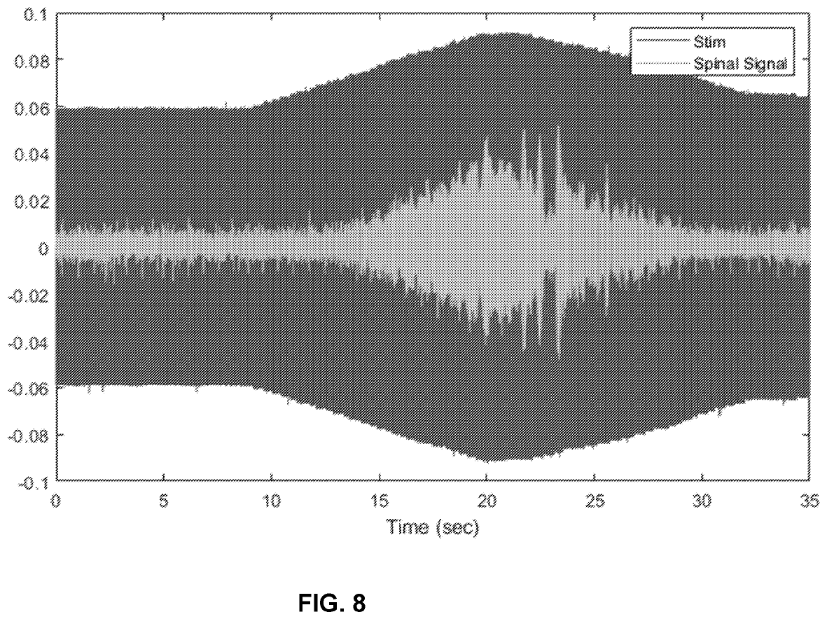

FIG. 8 is a time graph illustrating an example of adjusting the stimulation output and the effect the ECAP. The example of FIG. 8 shows that an increase the stimulation amplitude (darker signal) may cause an increase in the measured ECAP (light signal with a lower amplitude within the darker signal). Similarly, as the stimulation amplitude decreases, the measure ECAP may also decrease.

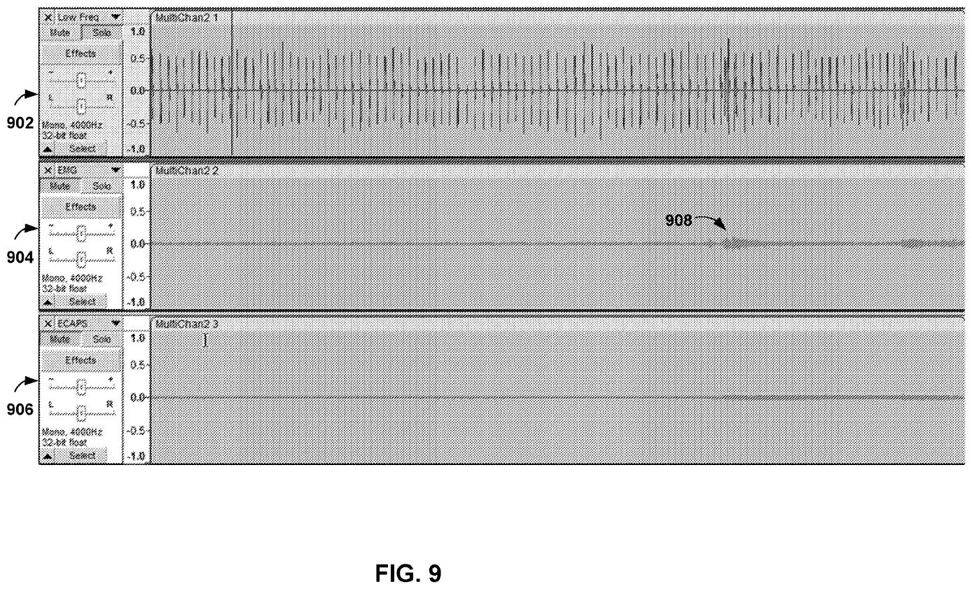

FIG. 9 is a time graph illustrating an example multi-channel representation of physiological signals. The example of FIG. 9 shows three signals along the same time scale, a low frequency signal 902, EMG 904 and ECAPS 906. A change in EMG 908, e.g., caused by an aggressor or other patient movement, may impact the ECAPS 906. In other examples, as described above, physiological signals other than EMG and ECAPS may be included in a multi-channel representation and converted to an audio signal.

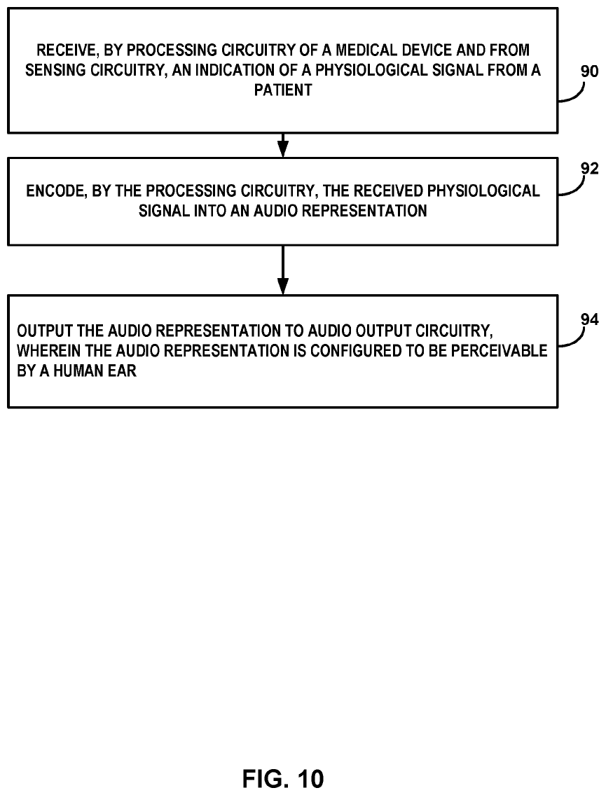

FIG. 10 is a flowchart illustrating an example operation of the audio generation system of this disclosure. Any processing circuitry described above in relation to FIGS. 1-4 may perform the functions in the blocks of FIG. 10, as well as one or more, portions of each block may be shared among the processing circuitry of separate computing devices. FIG. 10 will be described in terms of FIG. 3 to simplify the description.

For example, processing circuitry 350 may receive from sensing circuitry of IMD 110 of FIG. 1, e.g., via communication circuitry 356, an indication of a physiological signal from a patient (90). In some examples, the physiological signal may include an ECAP or some other physiological element of the physiological signal.

Processing circuitry 350 may receive a selection of at least one physiological element of the plurality of physiological elements, e.g., from a user via input controls 372 of user interface 354. In response to the received selection, processing circuitry 350 may encode the selected at least one physiological element of the physiological signal into an audio representation (92) such as by transforming the received indication into a sound, or by amplifying the physiological element, or some other encoding described above, for example in FIG. 4.

Processing circuitry 350 may further control audio output circuitry to output the audio representation, wherein the audio representation is configured to be perceivable by a human ear (94). In some examples audio output circuitry is directly connected to the same device as processing circuitry 350. In other examples, the audio output circuitry may be one or more separate speakers, headphones or similar output devices connected via communication circuitry 356.

In one or more examples, the functions described above may be implemented in hardware, software, firmware, or any combination thereof. For example, the various components of FIGS. 1-4, such as servers 160, processing circuitry 210, processing circuitry 350 and signal processing circuitry 420 may be implemented in hardware, software, firmware, or any combination thereof. If implemented in software, the functions may be stored on or transmitted over, as one or more instructions or code, a computer-readable medium and executed by a hardware-based processing unit. Computer-readable media may include computer-readable storage media, which corresponds to a tangible medium such as data storage media, or communication media including any medium that facilitates transfer of a computer program from one place to another, e.g., according to a communication protocol. In this manner, computer-readable media generally may correspond to (1) tangible computer-readable storage media which is non-transitory or (2) a communication medium such as a signal or carrier wave. Data storage media may be any available media that can be accessed by one or more computers or one or more processors to retrieve instructions, code and/or data structures for implementation of the techniques described in this disclosure. A computer program product may include a computer-readable medium.

The term “non-transitory” may indicate that the storage medium is not embodied in a carrier wave or a propagated signal. In certain examples, a non-transitory storage medium may store data that can, over time, change (e.g., in RAM or cache). By way of example, and not limitation, such computer-readable storage media, may include random access memory (RAM), read only memory (ROM), programmable read only memory (PROM), erasable programmable read only memory (EPROM), electronically erasable programmable read only memory (EEPROM), flash memory, a hard disk, a compact disc ROM (CD-ROM), a floppy disk, a cassette, magnetic media, optical media, or other computer readable media. In some examples, an article of manufacture may include one or more computer-readable storage media.

Also, any connection is properly termed a computer-readable medium. For example, if instructions are transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. It should be understood, however, that computer-readable storage media and data storage media do not include connections, carrier waves, signals, or other transient media, but are instead directed to non-transient, tangible storage media. Combinations of the above should also be included within the scope of computer-readable media.

Instructions may be executed by one or more processors, such as one or more DSPs, general purpose microprocessors, ASICs, FPGAs, or other equivalent integrated or discrete logic circuitry. Accordingly, the term “processor” and “processing circuitry,” as used herein, may refer to any of the foregoing structure or any other structure suitable for implementation of the techniques described herein. Also, the techniques could be fully implemented in one or more circuits or logic elements.

The techniques of this disclosure may be implemented in a wide variety of devices or apparatuses, including, an integrated circuit (IC) or a set of ICs (e.g., a chip set). Various components, modules, or units are described in this disclosure to emphasize functional aspects of devices configured to perform the disclosed techniques, but do not necessarily require realization by different hardware units. Rather, as described above, various units may be combined in a hardware unit or provided by a collection of interoperative hardware units, including one or more processors as described above, in conjunction with suitable software and/or firmware.

The techniques of this disclosure may also be described in the following examples.

Example 1: A system comprising a memory; and processing circuitry operatively coupled to the memory, the processing circuitry configured to: receive, from sensing circuitry, information representative of a physiological signal from a patient, wherein the information represents a plurality of physiological elements contained in the physiological signal; receive a selection of at least one physiological element of the plurality of physiological elements; encode the selected at least one physiological element of the physiological signal into an audio representation; and control audio output circuitry to output the audio representation, wherein the audio representation is configured to be perceivable by a human ear.

Example 2: The system of example 1, wherein the processing circuitry is operatively coupled to input controls of a user interface, wherein the processing circuitry is configured to receive, via the input controls, a user input, wherein the audio representation is a first audio representation, wherein, responsive to the user input, the processing circuitry is configured to encode the received physiological signal into a second audio representation according to the user input.

Example 3: The system of example 2, wherein the sensing circuitry is a component of an implantable medical device (IMD), wherein, responsive to the user input, the processing circuitry is further configured to change one or more operating parameters of the IMD.

Example 4: The system of example 3, wherein the operating parameters comprise one of an open loop configuration or a closed loop configuration.

Example 5: The system of any of examples 3 and 4, wherein the operating parameters comprise one or more of: an electrode selection, a sensing window size, a blanking window size, a stimulation output amplitude, or a control pulse parameter.

Example 6: The system of any of examples 2 through 5, wherein to encode the selected physiological element of the received physiological signal the processing circuitry is configured to apply filtering to the physiological signal.

Example 7: The system of example 6, wherein to the processing circuitry is configured to apply the filtering by at least filtering the physiological signal using one or more of a bandpass filter, high pass filter, low pass filter, exponential filtering, digital filtering, adaptive filtering, or time window blanking.

Example 8: The system of example 7, wherein the filtering comprises the exponential filtering, and wherein the exponential filtering is configured to remove a stimulation artifact.

Example 9: The system of any of examples 6 through 8, wherein the processing circuitry is configured to apply the filtering by at least focusing the audio representation on specified physiological elements of the physiological signal.

Example 10: The system of any of examples 6 through 9, wherein the processing circuitry is configured to, responsive to the user input, change the filtering applied to the physiological signal.

Example 11: The system of any of examples 2 through 10, wherein the processing circuitry is configured to encode the selected physiological element by at least modulating an audio signal with the selected physiological element of the received physiological signal.

Example 12: The system of any of examples 2 through 11, wherein the processing circuitry is configured to encode the selected physiological element by at least amplifying the selected physiological element of the received physiological signal.

Example 13: The system of any of examples 2 through 12, wherein the processing circuitry is configured to encode the selected physiological element by at least transforming the selected physiological element of the received physiological signal into an audio signal, wherein the audio signal is configured to indicate the presence of the physiological element, a change in the physiological element or indicate characteristics of the physiological element.

Example 14: The system of any of examples 2 through 13, wherein the received physiological signal is a first physiological signal, wherein the processing circuitry is further configured to: receive from the sensing circuitry second information representative of a second physiological signal from the patient; combine the first physiological signal and the second physiological signal into the audio representation; responsive to the received selection of at the least one physiological element of the plurality of physiological elements, encode the first physiological signal with one or more of a temporally different or a spectrally different audio representation from the second physiological signal, wherein the different audio representation is configured to distinguish the first physiological signal from the second physiological signal.

Example 15: The system of example 14, wherein the first physiological signal and second physiological signal each comprise an ECAP evoked from a same target tissue as the first physiological signal, wherein the second physiological signal differs from the first physiological signal based on a first latency between a first stimulation pulse and the first physiological signal and a second latency between a second stimulation pulse and the second physiological signal.

Example 16: The system of any of examples 14 and 15, wherein to encode the spectrally different audio representation, the processing circuitry is configured to transform the first physiological signal with a different transform application than the second physiological signal, and wherein the spectrally different audio representation comprises a difference based on one or more of: timbre, frequency, amplitude, ringing, or modulation type.

Example 17: The system of any of examples 14 through 16, wherein the system comprises the audio output circuitry, and wherein the audio representation is configured to be output by the audio output circuitry on two or more virtual channels.

Example 18: The system of any of examples 14 through 17, wherein the selected physiological element of the first physiological signal further comprises any one or more of: local field potentials (LFP), electrically evoked compound action potential (EECAP), evoked resonant neural activity (ERNA), evoked compound action potential (ECAP), electromyogram (EMG), cardiac activity, patient respiration, impedance, and wherein the second physiological signal comprises one or more of: an ECAP, LFP, ERNA, EMG, cardiac activity, patient respiration, impedance, temperature, pressure, motion, posture, and activity.

Example 19: A method comprising receiving, by processing circuitry of a medical device and from sensing circuitry, information representative of a physiological signal from a patient, wherein the information represents a plurality of physiological elements contained in the physiological signal; receiving a selection of at least one physiological element of the plurality of physiological elements encoding, by the processing circuitry, the selected at least one physiological element of the physiological signal into an audio representation; and controlling audio output circuitry to output the audio representation, wherein the audio representation is configured to be perceivable by a human ear.

Example 20: A non-transitory computer-readable storage medium comprising receive, from sensing circuitry of a medical device, an indication of a physiological signal from a patient, wherein the physiological signal comprises an evoked compound action potential (ECAP); receive a selection of at least one physiological element of the plurality of physiological elements; encode the selected at least one physiological element of the physiological signal into an audio representation; and control audio output circuitry to output the audio representation, wherein the audio representation is configured to be perceivable by a human ear.

Various examples of the disclosure have been described. These and other examples are within the scope of the following claims.

1-14. (canceled)

15. A system comprising:
a memory; and
processing circuitry operatively coupled to the memory, the processing circuitry configured to:
receive, from sensing circuitry, information representative of a physiological signal from a patient, wherein the information represents a plurality of physiological elements contained in the physiological signal;
receive a selection of at least one physiological element of the plurality of physiological elements;
encode the selected at least one physiological element of the physiological signal into an audio representation; and
control audio output circuitry to output the audio representation, wherein the audio representation is configured to be perceivable by a human ear.

16. The system of claim 15,
wherein the processing circuitry is operatively coupled to input controls of a user interface,
wherein the processing circuitry is configured to receive, via the input controls, a user input,
wherein the audio representation is a first audio representation,
wherein, responsive to the user input, the processing circuitry is configured to encode the received physiological signal into a second audio representation according to the user input.

17. The system of claim 16,
wherein the sensing circuitry is a component of an implantable medical device (IMD),
wherein, responsive to the user input, the processing circuitry is further configured to change one or more operating parameters of the IMD.

18. The system of claim 17, wherein the operating parameters comprise one of an open loop configuration or a closed loop configuration.

19. The system of claim 17, wherein the operating parameters comprise one or more of: an electrode selection, a sensing window size, a blanking window size, a stimulation output amplitude, or a control pulse parameter.

20. The system of claim 16, wherein to encode the selected physiological element of the received physiological signal the processing circuitry is configured to apply filtering to the physiological signal.

21. The system of claim 20, wherein to the processing circuitry is configured to apply the filtering by at least filtering the physiological signal using one or more of a bandpass filter, high pass filter, low pass filter, exponential filtering, digital filtering, adaptive filtering, or time window blanking.

22. The system of claim 21, wherein the filtering comprises the exponential filtering, and wherein the exponential filtering is configured to remove a stimulation artifact.

23. The system of claim 20, wherein the processing circuitry is configured to apply the filtering by at least focusing the audio representation on specified physiological elements of the physiological signal.

24. The system of claim 20, wherein the processing circuitry is configured to, responsive to the user input, change the filtering applied to the physiological signal.

25. The system of claim 16, wherein the processing circuitry is configured to encode the selected physiological element by at least modulating an audio signal with the selected physiological element of the received physiological signal.

26. The system of claim 16, wherein the processing circuitry is configured to encode the selected physiological element by at least amplifying the selected physiological element of the received physiological signal.

27. The system of claim 16, wherein the processing circuitry is configured to encode the selected physiological element by at least transforming the selected physiological element of the received physiological signal into an audio signal, wherein the audio signal is configured to indicate the presence of the physiological element, a change in the physiological element or indicate characteristics of the physiological element.

28. The system of claim 16,
wherein the received physiological signal is a first physiological signal,
wherein the processing circuitry is further configured to:
receive from the sensing circuitry second information representative of a second physiological signal from the patient;
combine the first physiological signal and the second physiological signal into the audio representation;
responsive to the received selection of at the least one physiological element of the plurality of physiological elements, encode the first physiological signal with one or more of a temporally different or a spectrally different audio representation from the second physiological signal, wherein the different audio representation is configured to distinguish the first physiological signal from the second physiological signal.

29. The system of claim 28,
wherein the first physiological signal and second physiological signal each comprise an ECAP evoked from a same target tissue as the first physiological signal,
wherein the second physiological signal differs from the first physiological signal based on a first latency between a first stimulation pulse and the first physiological signal and a second latency between a second stimulation pulse and the second physiological signal.

30. The system of claim 28,
wherein to encode the spectrally different audio representation, the processing circuitry is configured to transform the first physiological signal with a different transform application than the second physiological signal, and
wherein the spectrally different audio representation comprises a difference based on one or more of: timbre, frequency, amplitude, ringing, or modulation type.

31. The system of claim 28,
wherein the system comprises the audio output circuitry, and
wherein the audio representation is configured to be output by the audio output circuitry on two or more virtual channels.

32. The system of claim 28,
wherein the selected physiological element of the first physiological signal further comprises any one or more of: local field potentials (LFP), electrically evoked compound action potential (EECAP), evoked resonant neural activity (ERNA), evoked compound action potential (ECAP), electromyogram (EMG), cardiac activity, patient respiration, or impedance, and
wherein the second physiological signal comprises one or more of: an ECAP, LFP, ERNA, EMG, cardiac activity, patient respiration, impedance, temperature, pressure, motion, posture, or activity.

33. A method comprising:
receiving, by processing circuitry of a medical device and from sensing circuitry, information representative of a physiological signal from a patient, wherein the information represents a plurality of physiological elements contained in the physiological signal;
receiving a selection of at least one physiological element of the plurality of physiological elements;
encoding, by the processing circuitry, the selected at least one physiological element of the physiological signal into an audio representation; and
controlling audio output circuitry to output the audio representation, wherein the audio representation is configured to be perceivable by a human ear.

34. A non-transitory computer-readable storage medium comprising instructions that, when executed, cause one or more processors of a computing device to:
receive from sensing circuitry of a medical device, information representative of a physiological signal from a patient, wherein the information represents a plurality of physiological elements contained in the physiological signal;
receive a selection of at least one physiological element of the plurality of physiological elements;
encode, by the processing circuitry, the selected at least one physiological element of the physiological signal into an audio representation; and
control audio output circuitry to output the audio representation, wherein the audio representation is configured to be perceivable by a human ear.
